(19) United States
(12) Patent Application Publication
(10) Pub. No.: US 2002/0165505 A1
(43) Pub. Date: Nov. 7, 2002

(54) Pervaporation separation of volatile siloxanes from emulsions

(76) Inventors: Ronald Paul Gee, Midland, MI (US); Burt Michael Wrolson, Midland, MI (US)

Correspondence Address: DOW CORNING CORPORATION CO1232, 2200 W. SALZBURG ROAD, P.O. BOX 994, MIDLAND, MI, 48686-0994, US

(21) Appl. No.: 09/797,296

(22) Filed: Mar. 1, 2001

(51) Int. Cl. A61K 31/665

(52) U.S. Cl. 604/289

(57) ABSTRACT

A method of removing volatile siloxane oligomers from emulsions containing siloxane polymers. The method can be applied to any emulsion containing siloxane polymers, but it's especially adapted for removing residual volatile siloxane oligomers from emulsions containing siloxane polymers prepared by emulsion polymerization of the volatile siloxane oligomers. In particular, volatile siloxane oligomers are removed from emulsions containing siloxane polymers by pervaporation.

### CROSS-REFERENCE TO RELATED APPLICATIONS

Not applicable. STATEMENT REGARDING FEDERALLY SPONSORED RESEARCH OR DEVELOPMENT

Not applicable.

### REFERENCE TO A MICROFICHE APPENDIX

Not applicable.

### FIELD OF THE INVENTION

This invention is directed to a method of removing volatile siloxane oligomers from emulsions containing siloxane polymers. While the method can be applied to any emulsion containing siloxane polymers, it is especially adapted for removing residual volatile siloxane oligomers from emulsions containing siloxane polymers prepared by emulsion polymerization of the volatile siloxane oligomers. In particular, volatile siloxane oligomers are removed from emulsions containing siloxane polymers by a pervaporation process.

### BACKGROUND OF THE INVENTION

U.S. Pat. No. 2,834,754 (May 13, 1958) describes a process for removing volatile organopolysiloxanes from high molecular weight organopolysiloxanes with a stripping gas such as steam, neon, nitrogen or argon, while kneading. According to that process, a Banbury mixer with sigma-type blades is used to remove octamethylcyclotetrasiloxane (D4) from a highly viscous masse or gummy elastic silicone solid. Stripping emulsions is not disclosed, however; and neither is pervaporation.

A process employing a stripping unit containing heated parallel plates is used in U.S. Pat. No. 4,096,160 (Jun. 20, 1978) to remove a steam heated mixture of hexamethylcyclotrisiloxane (D3), octamethylcyclotetrasiloxane, decamethylcyclopentasiloxane (D5), and dodecamethylcyclohexasiloxane (D6), from silanol terminated dimethylpolysiloxane fluids having a viscosity of 1,000-1,000,000 centistoke (mm²/s). Stripping emulsions is not disclosed, however; and neither is pervaporation.

Both U.S. Pat. No. 2,834,754 and U.S. Pat. No. 4,096,160 require specialized equipment for handling viscous polymers. Also, the rate of heat transfer is substantially reduced when processing such viscous polymers.

One known method for obviating processing difficulties associated with viscous polymers is to prepare and handle such polymers in the form of an aqueous emulsion. A process carried out in a heated flask is described in U.S. Pat. No. 4,600,436 (Jul. 15, 1986) for stripping emulsion polymerized polysiloxane emulsions of the cyclic siloxanes or other low molecular weight siloxanes used to prepare the emulsions. According to the '436 patent, emulsions stripped by such a batch process possess improved film properties. Pervaporation is not disclosed, however.

In another process described in U.S. Pat. No. 5,922,108 (Jul. 13, 1999), volatile organopolysiloxanes such as D4 are removed from a fluid stream such as air containing volatile organopolysiloxanes and a hydrocarbon such as methane or pentane, by passing the fluid stream through a column packed with dry soil. Stripping emulsions is not disclosed, however; and neither is pervaporation.

Pervaporation, however, is taught in U.S. Pat. No. 4,218,312 (Aug. 19, 1980); U.S. Pat. No. 6,039,878 (Mar. 21, 2000); U.S. Pat. No. 6,075,073 (Jun. 13, 2000); and U.S. Pat. No. 6,117,328 (Sep. 12, 2000); and while the '878 patent and the '073 patent remove volatile solvents from certain emulsions, neither patent describes the pervaporation of volatile siloxanes from emulsions containing siloxane polymers.

### BRIEF SUMMARY OF THE INVENTION

The invention relates to a method of removing volatile siloxane oligomers from aqueous emulsions containing siloxane polymers prepared by emulsion polymerization of the volatile siloxane oligomers by contacting an aqueous emulsion with a hydrophobic pervaporation membrane. The invention also relates to emulsions prepared according to this method. Such compositions are useful in treating surface and substrates such as hair, skin, paper, and textiles.

These and other features of the invention will become apparent from a consideration of the detailed description.

### BRIEF DESCRIPTION OF THE SEVERAL VIEWS OF THE DRAWING

Not applicable.

### DETAILED DESCRIPTION OF THE INVENTION

Pervaporation as that term is used herein, is understood to mean a process in which a multicomponent liquid stream, i.e., such as a silicone oil-in-water emulsion, is contacted with a membrane that preferentially permeates one or more of the components, i.e., such as a volatile siloxane oligomer like D4. As the feed liquid flows along the membrane surface, the preferentially permeated component, i.e., a volatile siloxane oligomer like D4, passes through the membrane as a vapor.

Transport through the membrane is induced by maintaining a vapor pressure on the permeate side of the membrane that is lower than the partial pressure of the feed liquid. The pressure difference is achieved by maintaining a vacuum, or by providing an inert purge such as air or nitrogen on the permeate side of the membrane. The permeate vapor can be condensed or released as needed. However, for purposes of this invention, it is preferred to condense the permeate. The condensate is removed as a two-phase mixture containing primarily volatile silicone oligomers and water. The residue, i.e., silicone oil-in-water emulsion, depleted of the permeating component, i.e., volatile silicone oligomers, exits on the feed side of the membrane.

It is generally desirable to separate the volatile siloxane oligomers from the water in the condensed permeate to facilitate the re-use or disposal of the materials. Suitable methods for separating volatile siloxane oligomers from water include passing the two-phase mixture through a separating device, such as a settling tank, cyclone, centrifuge, coalescer, separating membrane, or a combination of such devices.

While the pervaporation process has been applied to the removal of dissolved water from organic solvents, to the extraction of organic solvents from water, and to the separation of mixed organic solvents; it has not generally been applied to the separation of the more industrially significant organic/organic mixtures, nor has it been applied to the separation of multicomponent liquid streams containing organosilicon compounds.

The selectivity of pervaporation membranes varies considerably. Thus, while a silicone rubber membrane may be capable of removing a volatile organic component from a multicomponent liquid stream selectively, a crosslinked poly(vinyl alcohol) (PVA) membrane can remove only water from the same multicomponent liquid stream selectively. This difference occurs because silicone rubber is hydrophobic, permeating hydrophobic components preferentially; while PVA is hydrophilic, permeating hydrophilic components preferentially. Since the volatile siloxane oligomers of interest in this invention are hydrophobic, it is preferred to employ hydrophobic pervaporation membranes.

While the use of membranes prepared from polydimethylsiloxane rubber polymers is most preferred, any type of hydrophobic membrane which repels water can be employed, such as polyethylene, polypropylene, poly(1-trimethylsilyl-1-propyne), polyurethane, polybutadiene polyether imide, polyether block polymers, styrene butadiene rubber, nitrile butadiene rubber, ethene propene terpolymers, polychloroprene, chlorosulfonated polyethylene, polyethersulfone, polysilicone carbonate copolymers, fluoroelastomers such as polytetrafluoroethylene, plasticized polyvinyl chloride, cis-polybutadiene, cis-polyisoprene, poly(butene-1), polyester amide, nylon, and block copolymers of polyether and polyester.

According to the present invention, a batch method can be carried out, for example, by heating a silicone oil-in-water emulsion to a temperature of about 30-105° C., and circulating the silicone oil-in-water emulsion through a spirally-wound module containing a polydimethylsiloxane pervaporation membrane at a pressure in the range of about one atmosphere/1.013 bar/101.3 kPa to four atmosphere/4.052 bar/405.2 kPa. The pressure on the permeate side of the membrane is controlled at less than about 100 mm Hg/100 Torr to ensure that the volatile siloxanes are vaporized while passing through the membrane. Circulation through the module is continued until the desired degree of removal of volatile siloxanes from the silicone oil-in-water emulsion has been obtained, i.e., generally less than about 1.0 percent, preferably less than about 0.5 percent by weight of volatile siloxane based upon the weight of the silicone oil-in-water emulsion.

The present invention is not limited to a batch process. A continuous flow process method can be carried out, for example, by continuously feeding an emulsion to a heat exchange device, continuously feeding the heated emulsion to a pervaporation module or a plurality of pervaporation modules with sufficient membrane area to effect the desired separation in a single pass, continuously removing permeate material from the pervaporation module or plurality of pervaporation modules, and continuously removing the emulsion depleted of permeating component from the pervaporation module or plurality of pervaporation modules. Water or steam may be added to the emulsion as it passes through the pervaporation module or plurality of pervaporation modules, to replace water removed as permeate. Heat may be added to the emulsion as it passes through the pervaporation module or plurality of pervaporation modules, to replace latent heat lost by evaporation of water across the membrane.

Any of the known types of membrane modules can be used herein to carry out the process. For example, the module containing a large surface area of membrane can be a spirally wound module, a hollow fiber shell side feed module, a hollow fiber bore side feed module, a plate and frame module, or a tubular module. A single module can be used, or a plurality of modules can be connected in series or parallel.

The process is capable of functioning in a practical manner using emulsions containing a siloxane polymer with a viscosity of 10-100,000,000 centistoke (mm²/s).

The silicone oil-in-water emulsion can be an anionic, cationic, amphoteric, or a nonionic type of silicone oil-in-water emulsion, wherein the type of emulsion is determined by the type of surfactant present, including polymeric surfactants such as silicone polyethers and polyvinyl alcohol. It can be a silicone oil-in-water emulsion prepared by any technique including those processes which can be classified as being mechanical emulsification processes, i.e., European Published Application 463 431 (Jan. 2, 1992) and U.S. Pat. No. 5,763,505 (Jun. 9, 1998); suspension polymerization processes, i.e., as described for example by D. Huebner and J. Saam, in an article in Journal of Polymer Science, Polymer Chemistry Edition, Volume 20, Pages 3351-3368 (1982); or emulsion polymerization processes.

As a practical matter, the method according to the invention possesses the most commercial value with respect to silicone oil-in-water emulsions prepared by emulsion polymerization processes, since volatile siloxanes are used as oligomers in the emulsion polymerization process, and the products of emulsion polymerization as a consequence contain them as components.

As used herein, the term emulsion polymerization refers to any of the polymerization processes known in the art, as represented for example by processes such as described in US Patents 2891920 (Jun. 23, 1959), 3294725 (Dec. 27, 1966), 4999398 (Mar. 12, 1991), 5502105 (Mar. 26, 1996), 5661215 (Aug. 26, 1997), and European Patent Specification EP 0 459 500 B1 (Mar. 5, 1997).

These emulsion polymerization processes are typically carried out at a temperature in the range of 25-100° C., preferably 50-95° C., and involve opening of the ring of a volatile siloxane oligomer using an acid or a base catalyst in the presence of water. Upon opening of the ring, siloxanes with terminal hydroxy groups are formed. These siloxanes then react with one another by a condensation reaction to form the siloxane polymer.

A simplified representation of the process chemistry is shown below for a volatile siloxane oligomer such as octamethylcyclotetrasiloxane, in which Me represents CH₃. (Me₂SiO)₄ +H₂O+Catalyst→HOMe₂SiOMe₂SiOMe₂SiOSiMe₂OH→HOMe₂SiOMe₂SiOMe₂SiOSiMe₂OH+HOMe₂SiOMe₂SiOMe₂SiOSiMe₂OH→HOMe₂SiO(Me₂SiO)₆SiMe₂₀H+H₂O.

Siloxane polymers of higher molecular weight can be obtained by allowing this process to continue. The silicone emulsion polymerization process produces a silicone phase that contains a mixture of linear and cyclic polyorgansiloxanes.

Catalysts used in such processes include strong mineral acids such as hydrochloric acid; strong alkaline catalysts such as sodium hydroxide; quaternary ammonium hydroxides; surface active sulfonic acids such as dodecylbenzene sulfonic acid and the sodium salts thereof; silanolates; and organosilanolates. Other examples of suitable catalysts can be found in U.S. Pat. Nos. 2,891,920, 3,294,725, 4,999,398, 5,502,105, 5,661,215, and EP 0 459 500 B1.

Generally, volatile siloxane oligomers removed by this process are cyclic siloxane monomers of the formula

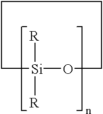

where each R is a saturated or unsaturated alkyl group of 1-6 carbon atoms, an aryl group of 6-10 carbon atoms, and n is 3-7. R can optionally contain a functional group which is unreactive in the ring opening and polymerization reaction.

Representative R groups are methyl, ethyl, propyl, phenyl, allyl, vinyl, and —RF′R′ is an alkylene group of 1-6 carbon atoms or an arylene group of 6-10 carbon atoms, and F is a functional group such as amine, diamine, halogen, carboxy, or mercapto. R can also be —R′F′R where R′ and R are described above and F′ is a non-carbon atom such as oxygen, nitrogen, or sulfur.

Volatile siloxanes oligomers of most interest herein include octamethylcyclotetrasiloxane (D4) and decamethylcyclopentasiloxane (D5). Silicone emulsions that can be treated according to the method of the invention include emulsions obtained by emulsion polymerization of only volatile cyclic siloxane oligomers or by emulsion polymerization of volatile cyclic siloxane oligomers in combination with alkoxysilanes. Suitable alkoxysilanes can be represented by the formulas R″Si(OR′″)₃, R″₂Si(OR′″)₂ or (R′″O)₄Si wherein R″ is either a neutral organic group such as an unsubstituted alkyl group CₐH₂ₐ₊₁ containing 1-12 carbon atoms or an aryl group such as phenyl, or a cationic organofunctional group such as an amino group. R′″ in hydrolyzable group (OR′″) in these formulas represents an alkyl group containing 1-6 carbon atoms. Silicone emulsions prepared with such alkoxysilanes generally contain 1-10 mole percent of R″ groups based on the total content of silicones in the emulsion.

The tetraalkoxysilanes (R′″O)₄Si are exemplified by tetramethoxysilane, tetraethoxysilane, tetrapropoxysilane, and tetrabutoxysilane.

Hydrolyzable or partially pre-hydrolyzed alkoxysilanes R″Si(OR′″)₃ with neutral organic groups R″ are exemplified by methyltrimethoxysilane (MTM), methyltriethoxysilane, ethyltrimethoxysilane, propyltrimethoxysilane, n-butyltrimethoxysilane, hexyltrimethoxysilane, octyltrimethoxysilane, octyltriethoxysilane, dodecyltrimethoxysilane, dodecyltriethoxysilane, and phenyltrimethoxysilane.

Hydrolyzable or partially pre-hydrolyzed alkoxysilanes R″Si(OR′″)₃ with cationic organofunctional groups R″ are exemplified by N-(2-aminoethyl)-3-aminopropyltrimethoxysilane, 3-aminopropyltrimethoxysilane, 3-aminopropylmethyldiethoxysilane, and n- cyclohexylaminopropyl methyldimethoxysilane.

Silicone emulsions that can be treated according to the method of the invention can contain anionic surfactants, including but not limited to, sulfonic acids and their salt derivatives. Some representative examples of anionic surfactants are alkali metal sulfosuccinates; sulfonated glyceryl esters of fatty acids such as sulfonated monoglycerides of coconut oil acids; salts of sulfonated monovalent alcohol esters such as sodium oleyl isethionate; amides of amino sulfonic acids such as the sodium salt of oleyl methyl tauride; sulfonated products of fatty acid nitrites such as palmitonitrile sulfonate; sulfonated aromatic hydrocarbons such as sodium alpha-naphthalene monosulfonate; condensation products of naphthalene sulfonic acids with formaldehyde; sodium octahydro anthracene sulfonate; alkali metal alkyl sulfates; ether sulfates having alkyl groups of eight or more carbon atoms such as sodium lauryl ether sulfate; and alkylaryl sulfonates having one or more alkyl groups of eight or more carbon atoms such as neutral salts of hexadecylbenzene sulfonic acid and C₂₀ alkylbenzene sulfonic acid.

Commercial anionic surfactants which can be useful in this invention include the sodium salt of dodecylbenzene sulfonic acid sold under the name SIPONATE DS-10 by Alcolac Inc., Baltimore, Md.; sodium n-hexadecyl diphenyloxide disulfonate sold under the name DOWFAX 8390 by The Dow Chemical Company, Midland, Mich.; the sodium salt of a secondary alkane sulfonate sold under the name HOSTAPUR SAS 60 by Clariant Corporation, Charlotte, N.C.; and linear alkyl benzene sulfonic acid sold under the name Bio-Soft S-100 by the Stepan Company, Northfield, Ill., which when neutralized functions as an anionic surfactant.

Silicone emulsions treated according to the method of the invention can contain cationic surfactants, including compounds containing quaternary ammonium hydrophilic moieties in the molecule which are positively charged, such as quaternary ammonium salts represented by R3R4R5R6N+X⁻ where R3 to R6 are alkyl groups containing 1-30 carbon atoms, or alkyl groups derived from tallow, coconut oil, or soy; and X is halogen, i.e., chlorine or bromine. Dialkyl dimethyl ammonium salts which can be used are represented by R7R8N+(CH₃)₂X⁻ where R7 and RB are alkyl groups containing 12-30 carbon atoms or alkyl groups derived from tallow, coconut oil, or soy; and X is halogen. Monoalkyl trimethyl ammonium salts which can be used are represented by R9N⁺(CH₃)₃X⁻ where R9 is an alkyl group containing 12-30 carbon atoms or an alkyl group derived from tallow, coconut oil, or soy; and X is halogen.

Representative quaternary ammonium salts are dodecyltrimethyl ammonium chloride/lauryltrimethyl ammonium chloride (LTAC), cetyltrimethyl ammonium chloride (CTAC), didodecyldimethyl ammonium bromide, dihexadecyldimethyl ammonium chloride, dihexadecyldimethyl ammonium bromide, dioctadecyldimethyl ammonium chloride, dieicosyldimethyl ammonium chloride, didocosyldimethyl ammonium chloride, dicoconutdimethyl ammonium chloride, ditallowdimethyl ammonium chloride, and ditallowdimethyl ammonium bromide. These and other quaternary ammonium salts are commercially available under names such as ADOGEN, ARQUAD, TOMAH, and VARIQUAT.

Silicone emulsions that can be treated according to the method of the invention can contain nonionic surfactants. Commercial types of nonionic surfactants can be exemplified by 2,6,8-trimethyl-4-nonyloxy polyethylene oxyethanols (6EO) and (10EO) sold under the names TERGITOL® TMN-6 and TERGITOL® TMN-10; alkyleneoxy polyethylene oxyethanol (C₁₁₋₁₅ secondary alcohol ethoxylates 7EO, 9EO, and 15EO) sold under the names TERGITOL® 15-S-7, TERGITOL® 15-S-9, TERGITOL® 15-S-15; other C₁₁₋₁₅ secondary alcohol ethoxylates sold under the names TERGITOL® 15-S-12, 15-S-20, 15-S-30, 15-S-40; and octylphenoxy polyethoxy ethanol (40EO) sold under the name TRITON® X-405. All of these surfactants are sold by Union Carbide Corporation, Danbury, Conn.

Other types of commercial nonionic surfactants are nonylphenoxy polyethoxy ethanol (10EO) sold under the name MAKON 10 by Stepan Company, Northfield, Ill.; polyoxyethylene 23 lauryl ether (Laureth-23) sold commercially under the name BRIJ 35L by ICI Surfactants, Wilmington, Del.; and RENEX 30, a polyoxyethylene ether alcohol sold by ICI Surfactants, Wilmington, Del. When preparing a silicone oil-in-water emulsion by an emulsion polymerization process, the presence of a nonionic surfactant is optional. However, when one is present, it is present in combination with an anionic or cationic surfactant.

Silicone emulsions that can be treated according to the method of the invention may contain a salt that is a product of the neutralization reaction used to deactivate the catalyst used in an emulsion polymerization reaction. The salt can be a simple compound such as sodium acetate formed by neutralization of sodium hydroxide with acetic acid after emulsion polymerization with a cationic surfactant. This is described in U.S. Pat. No. 5,661,215. The salt can be a more complex compound such as triethanolamine dodecylbenzene sulfonate formed by neutralization of dodecylbenzene sulfonic acid with triethanolamine, as also described in U.S. Pat. No. 5,661,215. Other examples include sodium chloride and triethanolamine chloride.

Most typically, emulsions prepared according to this invention contain a siloxane polymer concentration of about 10 to 70 percent by weight of the total emulsion, preferably about 25 to 60 percent by weight. While emulsions containing less than about 10 percent siloxane polymer content can be made, such emulsions hold little or no economic value. The surfactant is generally present at about 0.05 to 30 percent by weight of the total emulsion, preferably about 0.1 to 20 percent by weight. Water and optional salts constitute the balance of the emulsion to 100 percent.

The addition of a preservative after the pervaporation process may be desirable since emulsions are susceptible to microbiological contamination. Some representative preservatives include compositions such as formaldehyde; 1,3-dimethylol-5,5-dimethyl hydantoin, i.e., DMDM HYDANTOIN; 5-bromo-5-nitro-1,3-dioxane; methyl or propyl paraben; sorbic acid; imidazolidinyl urea; and KATHON CG (5-chloro-2-methyl-4-isothiazolin-3-one).

It is generally desirable to control the polysiloxane viscosity or molecular weight during the pervaporation operation. Establishment of such control measures is required because the viscosity of polysiloxanes can decrease significantly, or drift, during removal of volatile siloxane oligomers from silicone emulsions, particularly when using techniques such as represented by U.S. Pat. No. 4,600,436.

Two chemical reactions are believed to occur during a stripping operation as shown below.

≡SiOSi≡+H₂O→≡SiOH+≡SiOH Reaction 1

linear or branched polysiloxanes→linear or branched polysiloxanes+cyclic polysiloxanes Reaction 2

Reaction 1 is the depolymerization reaction, and it was found to proceed much faster than Reaction 2. It is believed that the high interfacial area between the silicone phase and the water phase contributes to the relatively high rate of Reaction 1. Therefore, to minimize the concentration of volatile siloxane oligomers in the finished emulsion while maximizing the viscosity of the polymer, it is desirable to minimize the amount of time required to effect the pervaporation operation, and to maintain the emulsion pH as close to 7 as possible.

### EXAMPLES

The following examples are set forth in order to illustrate this invention in more detail.

### Example 1

A cationic silicone oil-in-water emulsion was prepared according to the emulsion polymerization process described in European Patent EP 0 459 500 B1 (Mar. 5, 1997), using ARQUAD 16-29 as the cationic surfactant, and RENEX 30 as the nonionic surfactant. The cationic oil-in-water emulsion had a volume-weighted mean particle size of 0.137 micron/137 nanometer, as determined with a Model 150 MICROTRAC® Ultrafine Particle Analyzer manufactured by Honeywell Incorporated, Phoenix, Ariz. The siloxane polymer contained amine functionality, and the pH of the emulsion was about 7. The siloxane polymer in the cationic silicone oil-in-water emulsion had a viscosity of about 3,030 centistokes (mm²/s), which was measured using a Brookfield Model DV-II Viscometer equipped with a CP-52 spindle operating at 10 revolutions per minute. The concentration of octamethylcyclotetrasiloxane (D4) in the emulsion was about 2.2 percent by weight of the total weight of the emulsion, as determined by gas chromatography. The total silicone content of the emulsion was about 35 percent by weight of the total weight of the emulsion.

2500 gram of the emulsion were loaded into the feed tank of a laboratory scale pervaporation system sold under the name PerVap® by Membrane Technology & Research, Inc., Menlo Park, Calif. Systems such as this, and their details, are described in various United States Patents, including U.S. Pat. No. 5,030,356 (Jul. 9, 1991), U.S. Pat. No. 5,069,793 (Dec. 3, 1991), U.S. Pat. No. 526G206 (Nov. 30, 1993), U.S. Pat. No. 5,417,847 (May 23, 1995), and U.S. Pat. No. 5,538,640 (Jul. 23, 1996).

The emulsion was pumped through a single pervaporation membrane module and back to the feed tank at a rate of about 5.3 liters per minute. The pervaporation membrane module was spiral-wound. The pervaporation membrane was a composite having a support layer of polyvinylidene fluoride coated with a permselective layer of silicone rubber. The thickness of the permselective layer was about 10 microns. The total membrane area was about 0.2 m². The pervaporation module was fitted with an anti-telescoping device. Three 500 milliliter traps made of glass were located in the permeate line between the pervaporation module and a vacuum pump. Two of the traps were for collecting the permeate material, and one safety trap was provided to protect the vacuum pump. Each trap was suspended in a Dewar flask filled with liquid nitrogen. The temperature of the feed to the pervaporation module was controlled with a heat exchange system. The heat exchange system was activated, and the target feed temperature was set to 68° C. An isolation valve was located between the pervaporation module and the traps. The isolation valve was closed as the module feed temperature increased from ambient to the target temperature of 68° C.

When the module feed temperature was steady at approximately 68° C., the vacuum pump was activated. A pressure of 267-1200 pascal/2-9 mm Hg was maintained in the permeate line. The isolation valve was opened, and permeate material was collected in the traps. The traps were configured in a way that allowed one trap to collect permeate material, while the other trap was taken out of service to enable measurement of the permeate flux. The permeate flux was calculated by dividing the mass of permeate collected, by the time period over which the collection occurred. The mass of permeate was obtained by thawing the frozen permeate material, and then measuring the weight of liquid material.

The pervaporation process was allowed to proceed for five hours. After the elapse of each hour, a sample of the emulsion was obtained, the permeate flux was measured, and an amount of deionized water approximately equal to the mass of permeate collected over the hour, was added to the feed tank. The average permeate flux over the five hour period was about 2.1 grams per minute. The concentration of D4 in a number of the emulsion samples was determined by gas chromatography, and the resulting value was adjusted to a constant nonvolatile content basis. These values are shown in Table 1.

TABLE 1

| | Concentration of D4 in Total |
|---|---|
| Elapsed Time, | Emulsion, Percent (Normalized to a |
| Hour | Nonvolatile Content of 34.4 Percent) |
| 0 | 2.22 |
| 1 | 1.93 |
| 3 | 1.82 |
| 5 | 1.65 |

Table 1 shows that the concentration of D4 in the emulsion was reduced as a result of treatment with the pervaporation process.

### Example 2

An anionic silicone oil-in-water emulsion was prepared according the emulsion polymerization process described in U.S. Pat. No. 5,661,215 (Aug. 26, 1997), using BIO-SOFT S-100 as anionic surfactant and BRIJ 35 as nonionic surfactant. The anionic silicone oil-in-water emulsion had a volume-weighted mean particle size of 0.034 micron/34 nanometer, as determined with a Model 150 MICROTRAC® Ultrafine Particle Analyzer manufactured by Honeywell Incorporated, Phoenix, Arizona. The siloxane polymer contained (CH₃)₂SiO₂/₂ difunctional D units and CH₃SiO_{3/2} trifunctional T units. The pH of the emulsion was about 7. The siloxane polymer in the anionic silicone oil-in-water emulsion had a viscosity of about 840,000 centistoke (mm²/s), measured using a Brookfield Model HBDV-III Viscometer equipped with a CP-52 spindle operating at 0.5 revolutions per minute. The concentration of D4 in the emulsion was about 1.6 percent by weight of the total weight of the emulsion, as determined by gas chromatography. The total silicone content of the emulsion was about 25 percent by weight of the total weight of the emulsion.

2600 gram of the emulsion were processed generally as described in Example 1. The emulsion circulation rate through the pervaporation membrane module was about 4.9 liters per minute. The temperature of the emulsion fed to the pervaporation membrane module was about 85° C. A pressure of 933-2133 pascal/7-16 mm Hg was maintained in the permeate line. The average permeate flux over the five hour period was about 3.6 grams per minute. The concentration of D4 in a number of the emulsion samples was determined by gas chromatography, and the resulting value was adjusted to a constant nonvolatile content basis. These values are shown in Table 2.

TABLE 2

| | Concentration of D4 in Total |
|---|---|
| Elapsed Time, | Emulsion, Percent (Normalized to a |
| Hour | Nonvolatile Content of 38.8 Percent) |
| 0 | 1.59 |
| 1 | 1.28 |
| 3 | 0.91 |
| 5 | 0.73 |

Table 2 shows that the concentration of D4 in the emulsion was reduced as a result of treatment with the pervaporation process.

### Example 3

2700 gram of the anionic silicone oil-in-water emulsion of Example 2 were processed generally as described in Example 1. The emulsion circulation rate through the pervaporation membrane module was about 4.9 liters per minute. The temperature of the emulsion fed to the pervaporation membrane module was about 68° C. A pressure of 267-1200 pascal/2-9 mm Hg was maintained in the permeate line. The average permeate flux over the five hour period was about 2.0 grams per minute. The concentration of D4 in a number of the emulsion samples was determined by gas chromatography, and the resulting value was adjusted to a constant nonvolatile content basis. These values are shown in Table 3.

TABLE 3

| | Concentration of D4 in Total |
|---|---|
| Elapsed Time, | Emulsion, Percent (Normalized to a |
| Hour | Nonvolatile Content of 38.8 Percent) |
| 0 | 1.59 |
| 1 | 1.24 |
| 3 | 1.15 |
| 5 | 0.98 |

Table 3 shows that the concentration of D4 in the emulsion was reduced as a result of treatment with the pervaporation process.

A benefit of this invention is that it is often desirable to prepare silicone emulsions that contain low levels of volatile siloxane oligomers, because of certain environmental, health, and safety requirements, now mandated in many domestic and foreign jurisdictions.

The removal of volatile siloxane oligomers from emulsions is also a benefit to the extent that their removal and reuse prevents the loss of an otherwise valuable commodity, i.e., the volatile siloxane oligomer, in many applications where only the siloxane polymer has any real value in the application.

An additional benefit of this invention is that no foam control measure, i.e., antifoam compound or mechanical means of collapsing foam, is needed during processing. This is for the reason that gas and liquid are never present in the emulsion simultaneously. By contrast, it was found herein that it is often necessary to use an antifoam compound when removing volatile siloxane oligomers from silicone oil-in-water emulsions by steam stripping processes of the type described in U.S. Pat. No. 4,600,436. This benefit is significant, especially as it relates to the ability to obtain clear microemulsions. This is for the reason that addition of an antifoam compound even at very low levels, is generally a detriment to product clarity, as it necessarily introduces into the microemulsion a small population of large particles. Furthermore, the tendency to produce foam during stripping operations limits the flow rate of stripping gas that can be introduced into a batch stripper, and therefore limits the rate at which the volatile components are removed. Pervaporation, on the other hand, does not suffer from these disadvantages.

Finally, removal of the volatile siloxane oligomer from emulsions used in textile mills, paper printing facilities, and other manufacturing operations, is a benefit since it obviates the potential conversion of volatile siloxane oligomers to silica dust in pollution control equipment that operates at high temperature. Silica dust is known to foul certain pollution control equipment, thereby reducing the operating efficiency and increasing the maintenance costs of such equipment.

Emulsions prepared according to this invention are useful in paper coating, textile coating, and home care applications for delivering silicone polymers to various surfaces and substrates. They can also be used to deliver silicone polymers of tailored Theological properties to the human body, i.e., as in shampoo bases to provide styling and conditioning benefits to hair, or as a delivery mechanism for use in the care of skin.

Compositions found to be most useful according to this invention generally comprise emulsions and microemulsions containing the siloxane polymer having an average particle diameter of less than about 1 micron/1,000 nanometer, and less than about 0.14 micron/140 nanometer, respectively.

Other variations may be made in compounds, compositions, and methods described herein without departing from the essential features of the invention. The embodiments of the invention specifically illustrated herein are exemplary only and not intended as limitations on their scope except as defined in the appended claims.

1. A method of removing volatile siloxanes from an aqueous emulsion containing siloxane polymers and volatile siloxanes comprising contacting the aqueous emulsion with a hydrophobic pervaporation membrane.

2. A method according to claim 1 in which the volatile siloxanes comprise cyclic siloxanes of the formula 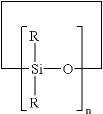
where n is 3-7; and each R is independently (i) a saturated or unsaturated alkyl group of 1-6 carbon atoms, (ii) an aryl group of 6-10 carbon atoms, (iii) the group —R′F where R′ is an alkylene group of 1-6 carbon atoms or an arylene group of 6-10 carbon atoms, and F is an amine, diamine, halogen, carboxy, or mercapto group, or (iv) the group —R′F′R where R′ and R are as described previously, and F′ is a non-carbon atom such as oxygen, nitrogen, or sulfur.

3. A method according to claim 2 in which the cyclic siloxanes comprise hexamethylcyclotrisiloxane, octamethylcyclotetrasiloxane, decamethylcyclopentasiloxane, dodecamethylcyclohexasiloxane, or mixtures thereof.

4. An emulsion prepared according to the method defined in claim 1.

5. A method of treating a surface or substrate selected from the group consisting of hair, skin, paper, and textile, comprising applying to the surface or substrate the emulsion prepared according to the method defined in claim 1.

6. A method of removing volatile siloxane oligomers from an aqueous emulsion containing siloxane polymers and volatile siloxane oligomers prepared by emulsion polymerization of the volatile siloxane oligomers comprising contacting the aqueous emulsion with a hydrophobic pervaporation membrane.

7. A method according to claim 6 in which the volatile siloxane oligomers comprise cyclic siloxane monomers of the formula 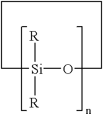
where n is 3-7; and each R is independently (i) a saturated or unsaturated alkyl group of 1-6 carbon atoms, (ii) an aryl group of 6-10 carbon atoms, (iii) the group —R′F where R′ is an alkylene group of 1-6 carbon atoms or an arylene group of 6-10 carbon atoms, and F is an amine, diamine, halogen, carboxy, or mercapto group, or (iv) the group —R′F′R where R′ and R are as described previously, and F′ is a non-carbon atom such as oxygen, nitrogen, or sulfur.

8. A method according to claim 7, in which the cyclic siloxanes comprise hexamethylcyclotrisiloxane, octamethylcyclotetrasiloxane, decamethylcyclopentasiloxane, dodecamethylcyclohexasiloxane, or mixtures thereof.

9. An emulsion prepared according to the method defined in claim 6.

10. A method of treating a surface or substrate selected from the group consisting of hair, skin, paper, and textile, comprising applying to the surface or substrate the emulsion prepared according to the method defined in claim 6.
